(19) United States
(12) Patent Application Publication
(10) Pub. No.: US 2009/0182435 A1
(43) Pub. Date: Jul. 16, 2009

(54) LIMB PROSTHESIS

(75) Inventor: Louis J. HABERMAN, Denville, NJ (US)

Correspondence Address: HARNESS, DICKEY & PIERCE, P.L.C., P.O. BOX 828, BLOOMFIELD HILLS, MI, 48303, US

(73) Assignee: ENGINEERED SILICONE PRODUCTS, LLC, Newton, NJ (US)

(21) Appl. No.: 12/354,198

(22) Filed: Jan. 15, 2009

Related U.S. Application Data
(60) Provisional application No. 61/021,157, filed on Jan. 15, 2008.

(51) Int. Cl. A61F 2/80

(52) U.S. Cl. 623/36

(57) ABSTRACT

A liner of a limb prosthesis liner includes a main body portion, an open proximal end, and a closed distal end. The liner further includes a sealing rib. The sealing rib has a first portion spiraling around the liner.

### CROSS-REFERENCE TO RELATED APPLICATIONS

This application claims priority to U.S. Provisional Patent Application No. 61/021,157 filed 15 Jan. 2008, which application is herein expressly incorporated by reference.

### FIELD

The present teachings generally relate to prostheses for limbs such as arms and legs. The present teachings more particularly relate to a limb prosthesis having a liner and a socket that cooperate to define a spiral path for the expulsion of air therebetween. The present teachings also more particularly relate to a method of providing an improved liner/socket interface and seal for a limb prosthesis.

### INTRODUCTION

Various prosthetic devices for legs and arms are known in the art.

Known limb prostheses may generally include a liner and a shell socket. The liner is attached to the severed limb and the shell socket may be secured to the liner with suction. The remainder of the prosthesis may be attached to the shell.

While significant advancements have been made in the field of limb prosthetics in recent years, a need remains in the art for improved methods of suspending them to the body.

### SUMMARY OF THE INVENTION

The present teachings provide a limb prosthesis having improved fit, suspension and comfort.

According to one particular aspect, the present teachings provide a liner of a limb prosthesis. The liner includes a main body portion, an open proximal end, and a closed distal end. The liner further includes a sealing rib. The sealing rib has a first portion spiraling around the liner.

According to another particular aspect, the present teachings provide a limb prosthesis having a liner and a hollow shell socket. The hollow shell socket has a proximal end and a closed distal end. The proximal end of the shell (socket) is open to receive the liner-limb combination. The shell socket further includes a valve proximate to the distal end which is required for releasing air trapped between the spiral rib of the liner and the inner socket. The liner includes a main body portion having a proximal end and a distal end. The liner further includes a sealing rib spiraling around the main body portion.

According to yet another particular aspect, the present teachings provide a method of securing a limb prosthesis to a patient. The method includes securing a liner to the patient. The liner includes a main body portion having a proximal end and a distal end. The liner further including a sealing rib spiraling around the main body portion. The method additionally includes attaching a hollow shell to the liner. The hollow shell includes a distal end and a proximal end. The shell further including a valve proximate the distal end. The method further includes releasing air between the liner and the hollow shell through the valve to create a suction seal therebetween.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

The present invention will become more fully understood from the detailed description and the accompanying drawings, wherein:

FIG. 1 is perspective, environmental view illustrating a liner and shell of a limb prosthesis according to the present teachings, the liner and shell shown operatively associated with a patient.

FIG. 2 is a perspective, environmental view similar to FIG. 1, illustrating the shell socket removed from the liner.

FIG. 3 is a cross-sectional view taken through a sealing rib of the shell socket.

FIG. 3A is a cross-sectional view similar to FIG. 3 illustrating an alternative sealing rib.

FIG. 3B is another cross-sectional view similar to FIG. 3 illustrating another alternative sealing rib.

FIG. 3C is another cross-sectional view similar to FIG. 3 illustration another alternative sealing rib.

FIG. 3D is another cross-sectional view similar to FIG. 3 illustration another alternative sealing rib.

### DESCRIPTION OF VARIOUS ASPECTS

The following description of various aspects of the present invention is merely exemplary in nature and is in no way intended to limit the invention, its application, or uses.

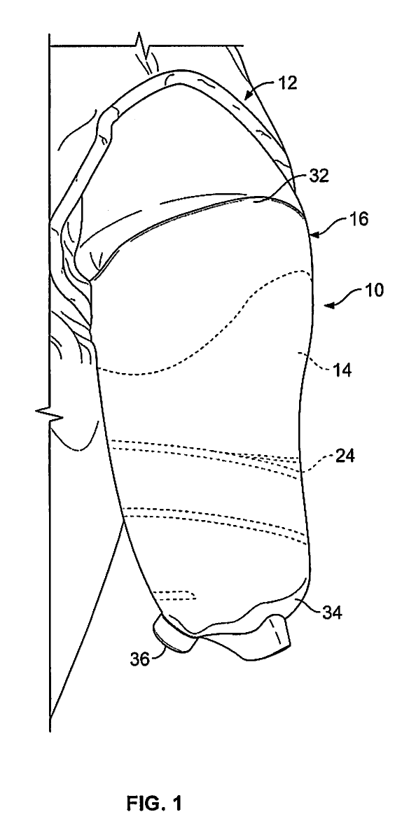

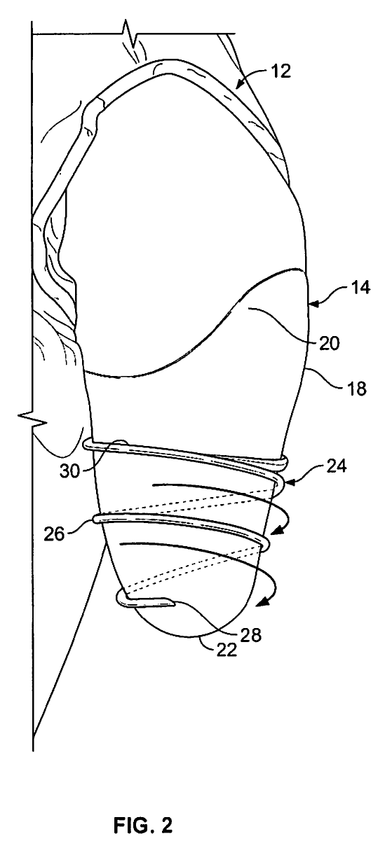

With general reference to FIGS. 1-3 of the drawings, a sub-assembly of a limb prosthesis constructed in accordance with the present teaching is illustrated and generally identified at reference character 10. In FIGS. 1 and 2, the sub-assembly is shown operatively associated with a patient 12. As particularly shown in the drawings, the present teachings may be used with an upper arm prosthesis. It will be appreciated, however, that the present teachings are not so limited. In this regard, the present teachings have application for any type of limb prosthesis. For example, the present teachings may alternatively be used in connection with above the knee prostheses, below the knee prostheses, and other prostheses.

The sub-assembly 10 may generally include a liner 14 and a shell 16. FIG. 1 illustrates the liner 14 and the shell 16 operatively associated with the patient 12. FIG. 2 illustrates the shell 16 removed from the liner 14. It will be understood by those skilled in the art that attachment of the remainder of the prosthesis (not shown) is conventional insofar as the present disclosure is concerned.

The liner 14 may be unitarily formed to define a main body portion 18 having a proximal end 20 and a distal end 22. The main body portion 18 is hollow and the proximal end 20 is open for attachment to the patient 12. The distal end 22 is closed.

The liner 14 may further include a sealing rib 24. The sealing rib 24 may be unitarily formed with the remainder of the liner 14. The sealing rib 24 may include a first portion 26 that spirals around the main body portion 18. The sealing rib 24 may terminate distally at an end 28 that is spaced from a reminder of the sealing rib 24. The sealing rib 24 may be initially constructed with the remainder of the liner 14 of silicone or other suitable material.

The sealing rib 24 may further include a second portion 30. The second portion 30 will circumscribe the main body portion 18 and may generally reside in a plane. The plane may be generally perpendicular to a long axis of the liner 14. The first and second portions 26 and 30 of the sealing rib 24 may be continuous such that the first portion 26 is simply an extension of the second portion 30 after the second portion circumscribes the main body portion 18. Alternatively, the first portion 26 and the second portion 30 may be spaced from one another.

The liner 14 may be formed of a silicone material though a liquid injection-molding process utilizing a series of mold sizes. For certain applications, the liner 14 may be formed of Shore A silicone in durometers of “0” to “20”. For other applications, the liner may be formed utilizing a Shore 00 (gel class) in durometers varying from ““25” to “40”.

The spiral portion 26 of the sealing rib 24 may include at least one ring extending about the main body portion 18. In the embodiment illustrated, the spiral portion 26 includes at least approximately two rings extending about the main body portion 18. Liners for larger and longer applications (e.g., those used for above the knee amputees), may have more rings.

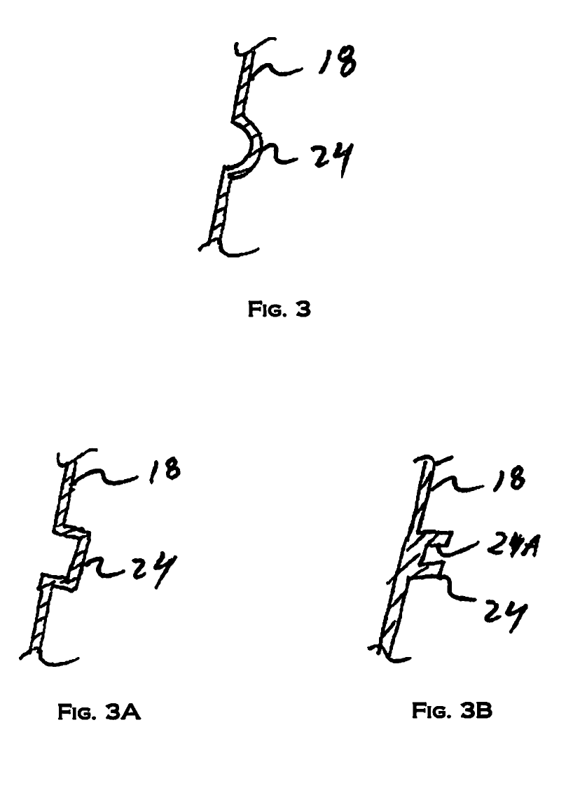

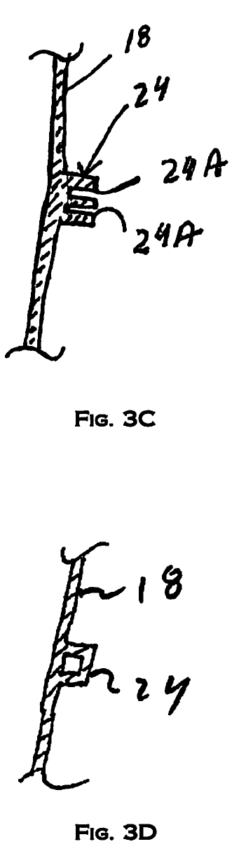

The sealing rib 24 may have a height and a width. The height may be between approximately 1/16″ and approximately 3/16″. The width may be between approximately ⅛″ and approximately 3/16″. It will be understood that these dimensions are merely exemplary and may vary depending on the material and type of application. As particularly shown in the cross-sectional view of FIG. 3, the sealing rib 24 may have a rounded radius. The geometry of the inflatable rib may depart from that shown in FIG. 3D.

With reference to FIGS. 3A-3D, alternative sealing ribs 24 are illustrated. In general, the sealing rib 24 may have an alternative geometry. For example, the sealing rib 24 is shown in FIG. 3A to have a generally square cross section. As shown particularly in FIGS. 3B and 3C, the sealing rib 24 may define one or more channels 24A. A single channel 24A is shown in FIG. 3B. A sealing rib 24 with a pair of channels 24A is shown in FIG. 3C. As shown in FIG. 3D, the sealing rib 24 may be a closed structure and may be inflatable. In this regard, a portion of the sealing rib 24 may be inflatable to increase/decrease sealing capability. The inflatable portion of the sealing rib 24 may be the uppermost portion.

The liner 14 may have a tapered proximal edge. As the liner terminates within the shell 12, contact pressure is exerted on the entire liner 14. Without the tapered proximal edge, the liner 14 may otherwise leave a marked depression and redness on the skin (referred to in the art as a line of demarcation). Furthermore, high shear forces will develop at the interface between the proximal edge of the liner and the skin. According to one particular application, the body of the liner 14 may have a thickness of approximately 6.0 mm to approximately 12.0 mm and the proximal edge may taper to approximately 1.00 mm to approximately 2.00 mm.

The shell 12 may be hollow shell having a proximal end 32 and a distal end 34. The proximal end 32 may be open to receive the liner 14. The shell 12 may include a valve 36 proximate to the distal end 34 for releasing air from an interior of the hollow shell 12. The valve 36 may be a conventional one-way valve that functions to permit art to vent from the between the shell 12 and the liner 14 and includes a button that may be depressed to reintroduce air (e.g., for removal of the shell 12).

In use, the liner 14 may be secured to the patient in a generally conventionally manner. The hollow shell 12 is inserted over the liner 14. The shell 12 may slightly compress the sealing rib 24 to provide a seal between the rib 24 and the shell 12. In certain applications, a cream (e.g., Nivea® or other) may be lightly applied to the sealing rib 24 prior to attachment of the shell 12 (e.g., wet fit). As the shell 12 is pulled into place, a seal is created at the second portion 30 of the sealing rib 24. Air trapped between the liner 14 and the shell 12 may follow a spiral path (see the arrows of FIG. 2) defined by the first portion 26 of the sealing rib 24 and exit through the valve 36.

As particularly compared to a single, horizontal suction rib (or seal), the spiral seal allows air to be evacuated throughout much of its length. The suction seal effect extends over a far greater surface area of the liner/limb elements. In addition, the spiral seal created by the present teachings provides greater suspension of the liner/limb to the socket as its higher seal secures more proximal regions of the socket to it and therefore results in improved overall prosthetic control.

While specific examples have been described in the specification and illustrated in the drawings, it will be understood by those skilled in the art that various changes may be made and equivalence may be substituted for elements thereof without departing from the scope of the present teachings as defined in the claims. Furthermore, the mixing and matching of features, elements and/or functions between various examples may be expressly contemplated herein so that one skilled in the art would appreciate from the present teachings that features, elements and/or functions of one example may be incorporated into another example as appropriate, unless described otherwise above. Moreover, many modifications may be made to adapt a particular situation or material to the present teachings without departing from the essential scope thereof. Therefore, it may be intended that the present teachings not be limited to the particular examples illustrated by the drawings and described in the specification as the best mode of presently contemplated for carrying out the present teachings but that the scope of the present disclosure will include any embodiments following within the foregoing description and any appended claims.

What is claimed is:

1. A liner of a limb prosthesis, the liner comprising:
an open proximal end;
a closed distal end; and
a sealing rib, the sealing rib having a first portion spiraling around the liner.

2. The liner of a limb prosthesis of claim 1, wherein the liner is injection molded.

3. The liner of a limb prosthesis of claim 1, wherein the liner is constructed of a polymer.

4. The liner of a limb prosthesis of claim 1, wherein at least a portion of scaling rib is inflatable.

5. The liner of a limb prosthesis of claim 1, wherein the sealing rib terminates distally at an end, the end being spaced from the remainder of the sealing rib.

6. The liner of a limb prosthesis of claim 1, wherein the sealing rib further includes a second portion circumscribing the liner in a plane generally perpendicular to a long axis of the liner.

7. The liner of a limb prosthesis of claim 1, wherein the liner tapers proximate the proximal end.

8. The liner of a limb prosthesis of claim 1, wherein the sealing rib has a height of at least approximately 1/16″.

9. A limb prosthesis comprising:
a liner; and
a hollow shell having a proximal end and a distal end, the proximal end being open to receive the liner, the shell including a valve proximate to the distal end for releasing air from an interior of the hollow shell;
wherein the liner includes a main body portion having a proximal end and a distal end, the liner further including a sealing rib spiraling around the main body portion.

10. The limb prosthesis of claim 9, wherein the sealing rib terminates distally at an end, the end being spaced from the remainder of the sealing rib.

11. The limb prosthesis of claim 9, wherein the sealing rib further includes a second portion circumscribing the liner in a plane generally perpendicular to a long axis of the liner.

12. The limb prosthesis of claim 9, wherein the liner tapers proximate the proximal end.

13. The limb prosthesis of claim 9, wherein at least a portion of the sealing rib is inflatable.

14. The limb prosthesis of claim 9, wherein the shell compresses the sealing rib to create a seal between the sealing rib and the shell.

15. The limb prosthesis of claim 9, wherein the spiral includes at least two rings.

16. A method of securing a limb prosthesis to a patient, the method comprising:
securing a liner to a patient, the liner including a main body portion having a proximal end and a distal end, the liner further including a sealing rib spiraling around the main body portion;
attaching a hollow shell to the liner, the hollow shell including a distal end and a proximal end, the shell further including a valve proximate the distal end; and
releasing air between the liner and the hollow shell through the valve to create a suction seal therebetween.

17. The method of securing a limb prosthesis to a patient of claim 16, wherein attaching the hollow shell to the liner includes compressing the sealing rib with the shell to create a seal between the sealing rib and the shell.

18. The method of securing a limb prosthesis to a patient of claim 16, wherein attaching the hollow shell to the liner includes creating a seal between the liner and the shell in a plane generally perpendicular to a long axis of the liner.

19. The method of securing a limb prosthesis to a patient of claim 16, further comprising applying a cream to the sealing rib prior to attaching the hollow shell to the liner.

20. The method of securing a limb prosthesis to a patient liner of claim 16, further comprising inflating at least a portion of the sealing rib.
